SEQUENCE LISTING (3 sequences)

SEQ ID NO: 1 - protein, 121 aa, Artificial Sequence
  Synthetic
QVKLQQSGAELVRPGASVKLSCKASGYTFTNYWINWVKQRPGQGLEWIGNIYPSYIYTNYNQEFKDKVTLTVDESSSTAYMQLSSPTSEDSAVYYCTRSPYGYDEYGLDYWGQGTTVTVSS

SEQ ID NO: 2 - protein, 113 aa, Artificial Sequence
  Synthetic
DIELTQSPSSLTVTAGEKVTMNCKSSQSLLNSRNQKNYLTWYQQKPGQPPKLLIYWASTRESGVPDRFTGSGSGTDFTLTISSVQAEDLAVYYCQNDYVYPLTFGAGTKLEIK

SEQ ID NO: 3 - protein, 490 aa, Artificial Sequence
  Synthetic
MLLLVTSLLLCELPHPAFLLIPQVQLVQSGAEVKKPGASVKVSCKASGYTFTNYWINWVRQAPGQGLEWIGNIYPSYIYTNYNQEFKDKVTLTVDESTSTAYMELSSLRSEDTAVYYCTRSPYGYDEYGLDYWGQGTTVTVSSGGGGSGGGGSGGGGSDIQLTQSPSSLSASVGDRVTMTCKSSQSLLNTRNQKNYLTWYQQKPGKAPKLLIYWASTRESGVPSRFSGSGSGTDFTLTISSLQPEDFATYYCQNDYVYPLTFGQGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0310500 A1
(43) Pub. Date: Oct. 5, 2023

(54) Use of Immune Killer Cell Against Circulating Tumor Cells in Solid Tumor Treatment

(71) Applicant: Techno Craft Corporation Ltd., Niigata (JP)

(72) Inventors: Zhongxi Xie, Shanghai (CN); Qingshan Shen, Anhui (CN); Qinhua Zhou, Suzhou, Jiangsu (CN); Yunpeng Zhong, Suzhou, Jiangsu (CN); Suqiong Wang, Shanghai (CN)

(73) Assignee: Techno Craft Corporation Ltd., Niigata (JP)

(21) Appl. No.: 18/021,677

(22) PCT Filed: Jul. 13, 2021
(86) PCT No.: PCT/CN2021/106098

§ 371 Date: Feb. 16, 2023

(30) Foreign Application Priority Data
Sep. 3, 2020 (CN) 202010917702.1

(51) Int. Cl. A61K 35/17; A61P 35/00

(52) U.S. Cl. CPC A61K 35/17; A61P 35/00

(57) ABSTRACT

The use of an immune killer cell against circulating tumor cells in solid tumor treatment, which comprises transfusing the immune killer cell into peripheral blood of a subject to act on the circulating tumor cells in peripheral blood. In the use of a solid tumor treatment, the immune killer cell can act on the circulating tumor cells by means of transfusing an effective dose of the immune killer cell into the peripheral blood of the subject, which is beneficial for blocking solid tumor metastasis by means of inhibiting the colonization of circulating tumor cells in tissue and improving the survival period of the subject.

### TECHNICAL FIELD

The invention relates to the technical field of adoptive immunotherapy, in particular, the use of immune killer cells targeting circulating tumor cells in solid tumor treatment.

### BACKGROUND

Tumor cells released from solid tumors into peripheral blood are called circulating tumor cells (CTCs). For solid tumors, conventional surgery can effectively remove the primary tumor, but in the early stage of the primary tumor, CTCs may fall off and enter the blood or lymphatic system, CTCs with high activity and high metastatic potential can survive in the circulatory system, and then they may follow the circulation and colonize distant organs and tissues to form metastases, which greatly improves the recurrence rate of tumors. If the CTCs entering peripheral blood can be removed before they form metastases, not only it is expected to control the number of metastases and even prevent the formation of metastases with the combination of other clinical methods, but also it is helpful to reduce the difficulty of prognosis and treatment of tumor patients.

At present, most of the existing research on CTCs focuses on how to isolate and accurately identify CTCs in peripheral blood, etc., to evaluate the prognosis of tumor patients. Chinese patent application with the publication number CN112891659A discloses the use of a magnetic bead filtration device to separate and capture CTCs in peripheral blood to achieve the removal of blood CTCs. The CTCs throttled by the chip filter device involved in the application are limited, the duration is short, and there are problems such as residual magnetic beads, so it obviously does not have clinical application prospects. For clinical applications, it must be ensured that the processes of the detection and removal of CTCs are safe, effective, repeatable, and less harmful to the patients.

Cell therapy has achieved remarkable results in the treatment of hematological tumors, and there are already CAR-T cell therapy products for the treatment of adult patients with hematological tumors. However, the treatment of solid tumors, especially recurrent solid tumors, is still a tough nut to crack with cell therapy. Considering that CTCs may be released into peripheral blood during the treatment of solid tumors and induce the formation of metastases, CTCs have become the ideal target for cell therapy to cure solid tumors and prevent tumor recurrence.

Therefore, it is necessary to design a novel use of immune killer cells so as to avoid the above-mentioned problems in the existing solid tumor treatment process, inhibit tumor metastasis and achieve a radical cure for solid tumors.

### SUMMARY

The purpose of the present invention is to provide a use of immune killer cells targeting circulating tumor cells, which is beneficial to inhibit the colonization of circulating tumor cells in tissues, block solid tumor metastasis, and improve the survival period of subjects.

In order to achieve the above purpose, the use of the immune killer cells in the treatment of solid tumors comprises:
S0: providing a subject and immune killer cells, the subject's peripheral blood comprising circulating tumor cells; and
S1: injecting an effective dose of the immune killer cells into the peripheral blood of the subject, and slowing down or blocking metastasis of the solid tumors through specifically recognizing the circulating tumor cells and killing or eliminating the circulating tumor cells by the immune killer cells.

The use of the immune killer cells according to the present invention has the following beneficial effect: by injecting the immune killer cells into the peripheral blood of the subject to directly act on the circulating tumor cells and kill or eliminate them, it is beneficial to inhibit the colonization of the circulating tumor cells in tissues, block solid tumor metastasis, and improve the survival period of subjects.

Preferably, the immune killer cells are genetically modified immune cells.

More preferably, the immune cells are at least one of T cells, NK cells, NKT cells, dendritic cells, macrophages and B cells.

More preferably, the T cells are γδ T cells.

More preferably, the genetically modified immune cells are obtained by genetically modifying the immune cells with any one of chimeric antigen receptors and T cell receptors.

More preferably, the chimeric antigen receptor comprises an extracellular recognition region, a hinge region, a transmembrane region and an intracellular signal region, and the extracellular recognition region specifically recognizes any one of EpCAM, c-MET, CD47, Vimentin, E-cadherin, Cytokeratins, Zonula occludens, ESPR1, N-cadherin, Twist1, ZEB1, FGFR2111c, PLS3, ALDH1, CD44, GD2, GD3, Claudin18.2, Claudin6, and GD1a.

More preferably, the genetically modified immune cells are obtained by genetically modifying T cells with chimeric antigen receptors.

Preferably, the subject is an animal model of solid tumor metastasis or a patient with metastases, and the tumor metastasis patterns of both the animal model of solid tumor metastasis and the patient with metastases are hematogenous metastases.

More preferably, the step S0 further comprises introducing exogenous circulating tumor cells into a body of an experimental animal via any one of orthotopic transplantation, intravenous injection and subcutaneous transplantation, so as to establish the animal model of solid tumor metastasis.

More preferably, the step S1 is performed after the exogenous circulating tumor cells are introduced into the body of the experimental animal, and after obtaining a peripheral blood sample of the experimental animal and detecting that it contains the circulating tumor cells.

More preferably, the step S1 is performed after the exogenous circulating tumor cells are introduced into the body of the experimental animal to form tumor tissue.

More preferably, in the step S1, a cell suspension containing the immune killer cells is injected into the body of the experimental animal via intravenous injection, and the amount of the immune killer cells in the cell suspension injected each time is 1×10⁴/Kg−1×10⁸/Kg.

More preferably, the step S1 further comprises applying auxiliary medical means to the subject to effectively eliminate the solid tumors.

### BRIEF DESCRIPTION OF THE DRAWINGS

FIG. 1 is the flow chart of the use of the immune killer cells according to the examples of the present invention;

FIG. 2 is the result of the methodological experiment of the verification of the detection of CTCs utilizing a microfluidic chip in Example 1;

FIG. 3 is the flow analysis result of the reference sample of Example 2 and the EpCAM CAR-T positive rate detection, on the left side are unT cells, and on the right side are CAR-T cells;

FIG. 4 is the tumor cell survival rate of each group at the end point of the in vitro-simulated CTC killing experiment in Example 3;

FIG. 5 is the blood biochemical scatter plot comparison of the mice in each group on Day 17 in Example 4, wherein (a) alanine aminotransferase ALT (U/L) content; and (b) aspartate aminotransferase AST (U/L) content are shown;

FIG. 6 is a graph showing the comparison of the survival of human CD45+ and human CD3+ T cells in the peripheral blood of the mice in each group in Example 4;

FIG. 7 is a graph showing the changes in trend of animal body weight after administration in Example 4;

FIG. 8 is the growth trend graph of the tumor volumes of the mice in each group after administration in Example 4;

FIG. 9 is a comparison graph of the changes in the mean fluorescence values obtained by the statistics of the in vivo fluorescence images of the mice in each group after administration in Example 4;

FIG. 10 is a comparison graph of the in vivo fluorescence images of the mice in each group after administration in Example 4;

FIG. 11 is a comparison graph of (a) the fluorescence values of the isolated lungs and (b) the fluorescence values of the isolated livers of the experimental mice on Day 26 in Example 4;

FIG. 12 is a comparison graph of the fluorescence images of the isolated lungs and the isolated livers of the experimental mice on Day 26 in Example 4;

FIG. 13 shows the blood CTC contents of the experimental mice in each group on Day 26 in Example 4;

FIG. 14 is the blood biochemical scatter plot comparison of the mice in each group on Day 17 in Example 5, wherein (a) alanine aminotransferase ALT (U/L) content; and (b) aspartate aminotransferase AST (U/L) content are shown;

FIG. 15 is a graph showing the comparison of the survival of human CD45+ and human CD3+ T cells in the peripheral blood of the mice in each group in Example 5;

FIG. 16 is a graph showing the changes in trend of animal body weight after administration in Example 5;

FIG. 17 is a growth trend graph of the tumor fluorescence values of the mice in each group after administration in Example 5;

FIG. 18 is a comparison graph of the in vivo fluorescence images of the mice in each group after administration in Example 5;

FIG. 19 is a comparison graph of (a) the in vivo imaging fluorescence values of the isolated lungs and (b) the in vivo imaging fluorescence values of the isolated livers of the experimental mice on Day 27 in Example 5;

FIG. 20 is a comparison graph of the fluorescence images of the isolated lungs and the isolated livers of the experimental mice on Day 27 in Example 5; and

FIG. 21 shows the CTC contents in the blood samples of the experimental mice on Day 27 in Example 5.

### DETAILED DESCRIPTION

In order to make the purposes, technical solutions and advantages of the examples of the present invention clearer, the technical solutions in the examples of the present invention will be clearly and completely described below. Apparently, the examples described are part of rather than all of the examples of the present invention. Based on the examples of the present invention, all other examples obtained by those of ordinary skill in the art without inventive efforts shall fall within the protection scope of the present invention. Unless otherwise defined, technical or scientific terms used herein should have the ordinary meanings as understood by one of ordinary skill in the art to which this invention belongs. As used herein, “comprise” and the like mean that the element(s) or object(s) appearing before the word encompasses the element(s) or object(s) recited after the word and their equivalent(s), but do not exclude other element(s) or object(s).

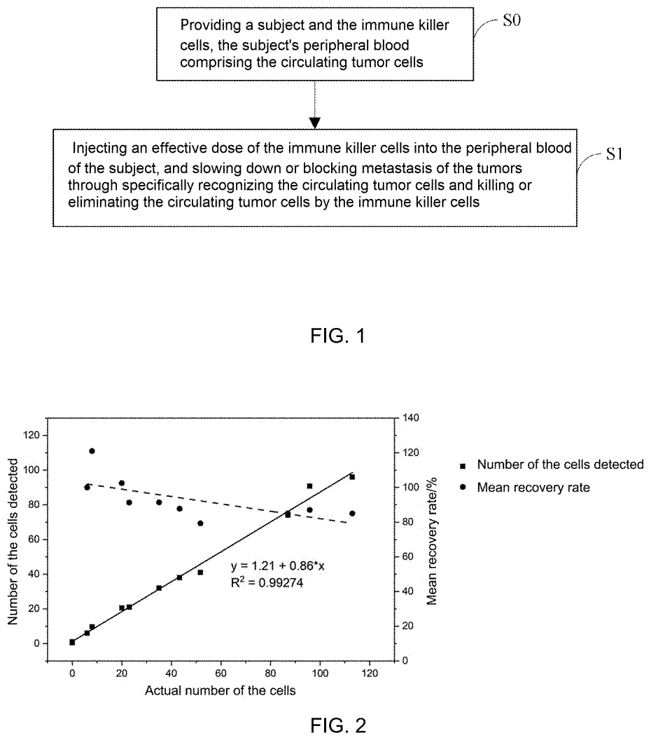

The examples of the present invention provide a use of immune killer cells targeting circulating tumor cells so as to help control or even prevent the formation of metastases in subsequent clinical applications, the use comprising (referring to FIG. 1):
S0: providing a subject and immune killer cells, the subject's peripheral blood comprising circulating tumor cells; and
S1: injecting an effective dose of the immune killer cells into the peripheral blood of the subject, and slowing down or blocking metastasis of the solid tumors through specifically recognizing the circulating tumor cells and killing or eliminating the circulating tumor cells by the immune killer cells.

In the examples of the present invention, by injecting the immune killer cells into the peripheral blood of the subject to directly act on the circulating tumor cells, it is possible to slow down or block the metastasis of the solid tumors through specifically recognizing the circulating tumor cells and killing or eliminating the circulating tumor cells by the immune killer cells.

In some examples of the present invention, the circulating tumor cells are derived from solid tumors.

Specifically, the antigen target of the circulating tumor cells is any one of EpCAM, c-MET, CD47, Vimentin, E-cadherin, Cytokeratins, Zonula occludens, ESPR1, N-cadherin, Twist1, ZEB1, FGFR2IIIc, PLS3, ALDH1, CD44, GD2, GD3, Claudin18.2, Claudin6 and GD1a.

In some examples of the present invention, the immune killer cells comprise specific polypeptides to induce apoptosis of the tumor cells by recognizing and binding the antigenic targets of the circulating tumor cells and triggering an immune response.

The meaning of the antigen target herein is: the binding site in the tumor cell that can interact with the specific polypeptide, specifically including biological macromolecules such as gene sites, receptors, enzymes, ion channels, and nucleic acids.

In some examples of the present invention, the specific polypeptide is a chimeric antigen receptor, and the immune killer cells are genetically modified immune cells. Specifically, the immune cells are at least one of T cells, NK cells, NKT cells, dendritic cells, macrophages and B cells.

In some examples of the present invention, the T cells are γδ T cells.

In some examples of the present invention, the genetically modified immune cells are obtained by genetically modifying the immune cells with any one of chimeric antigen receptors and T cell receptors. Chimeric Antigen Receptor (CAR) can target the antigen target of the circulating tumor cell.

In some examples of the present invention, the genetically modified immune cells are any one of CAR-T cells, CAR-NK cells and CAR-M cells.

In some examples of the present invention, the chimeric antigen receptor comprises a heavy chain variable region and a light chain variable region, and the sequence of the heavy chain variable region is a mutant sequence obtained through substitution of the sequence shown in SEQ ID No. 1, the sequence of the light chain variable region is a mutant sequence obtained through substitution or deletion of the sequence shown in SEQ ID No. 2.

In some examples of the present invention, the chimeric antigen receptor comprises an extracellular recognition region, a hinge region, a transmembrane region and an intracellular signaling region.

Specifically, the extracellular recognition region specifically recognizes any one of EpCAM, c-MET, CD47, Vimentin, E-cadherin, Cytokeratins, Zonula occludens, ESPR1, N-cadherin, Twist1, ZEB1, FGFR2IIIc, PLS3, ALDH1, CD44, GD2, GD3, Claudin18.2, Claudin6 and GD1a.

Specifically, the sequence of the hinge region is derived from at least one of CD8α, CD28, 4-1BB, ICOS, OX40, CD40, CD80 and IgG.

Specifically, the sequence of the transmembrane region is derived from at least one of CD8α, CD28, 4-1 BB, ICOS, OX40, CD40 and CD80.

Specifically, the sequence of the intracellular signal region is derived from at least one of CD8α, CD28, 4-1BB, ICOS, OX40, CD40, CD80, DAP10, DAP12, CD3 and CD3e.

In some examples of the present invention, the genetically modified immune cells are obtained by genetically modifying T cells with chimeric antigen receptors.

In some examples of the present invention, the subject is an animal model of solid tumor metastasis or a patient with metastases, and the tumor metastasis patterns of both the animal model of solid tumor metastasis and the patient with metastases are hematogenous metastases.

In some examples of the present invention, the step S0 further comprises introducing exogenous circulating tumor cells into a body of an experimental animal via any one of orthotopic transplantation, intravenous injection and subcutaneous transplantation, so as to establish the animal model of solid tumor metastasis.

Specifically, the exogenous circulating tumor cells are derived from a human solid tumor cell line or human solid tumor tissue.

Further, the step S1 is performed after the exogenous circulating tumor cells are introduced into the body of the experimental animal, and after obtaining a peripheral blood sample of the experimental animal and detecting it to determine the number of the circulating tumor cells contained in the peripheral blood sample.

Further, a cell suspension containing the immune killer cells is injected into the body of the experimental animal via intravenous injection, and the amount of the immune killer cells in the cell suspension injected each time is 1×10⁴/Kg−1×10⁸/Kg.

In some examples of the present invention, the step 51 further comprises applying auxiliary medical means to the subject to effectively eliminate solid tumors.

The use and beneficial effects of the immune killer cells are described in detail below through specific examples.

### Example 1

This example provides the verification of the relevant methodology for detecting CTCs using a microfluidic chip, including procedures of experimental modeling and detection with the chip, to prove that the microfluidic chip used in the present invention can sensitively and accurately detect the tumor cells in a blood sample.

In Example 1, anti-EpCAM capture antibody was from R&D System, and the product number was BAF960; serum protein was from Sangon Biotech, and the product number was A600332-0025; TritonX-100 was from Sigma, and the product number was X100-500ML; DAPI solution was from Invitrogen, and the product number was D1306.

The specific steps of the experimental modeling were:

the experimental blood samples were divided into a control group and an experimental group, and the blood samples of the experimental group had tumor cell contents of 5, 25, 50 or 100 cells/ml, with 2 blood samples for each content; and the blank control group was obtained by adding 10 microliters of phosphate buffered saline into 1 ml of healthy whole blood.

The preparation for each blood sample in the experimental group was:

human colorectal cancer cell suspension 1, human colorectal cancer cell suspension 2, human colorectal cancer cell suspension 3 and human colorectal cancer cell suspension 4 were composed of HCT116 cells in an amount of 1×10⁴/ml, 5×10³/ml, 2.5×10³/ml and 5×10²/ml, respectively, and phosphate buffered saline. 10 μl of each of the above suspensions was taken and the cells were counted under a microscope. After the amount of the cells in each suspension was determined, the suspension was centrifuged to let the cells into a centrifuge tube, and 1 ml of healthy whole blood was added to each centrifuge tube to obtain the blood sample containing the tumor cells for the experimental group, and the establishment of the model of the relevant methodology for detecting CTCs using the microfluidic chip was completed.

The specific steps of the detection with the chip were:

the microfluidic chip was incubated with anti-EpCAM capture antibody overnight, then washed with phosphate buffered saline, and then the chip was blocked with serum protein for 1 hour, washed again with phosphate buffered saline, and stored at low temperature and ready for use;

the experimental blood sample was slowly injected into the microfluidic chip, and after the injection, the microfluidic chip was slowly rinsed with phosphate buffered saline, and 4% paraformaldehyde solution was added into the microfluidic chip to fix the cells, and then the microfluidic chip was washed with phosphate buffered saline containing Tween20;

TritonX-100 was added into the microfluidic chip, and after incubation for 5 minutes, the microfluidic chip was washed with phosphate buffered saline containing Tween20, and then the microfluidic chip was blocked with serum protein again for 1 hour, and then the microfluidic chip was washed with phosphate buffered saline containing Tween20;

a primary antibody solution containing anti-CK antibody and anti-CD45 antibody was added into the microfluidic chip, and the chip was incubated for 1 hour, and then the microfluidic chip was washed with phosphate buffered saline containing Tween20; then a secondary antibody solution was added into the chip and the chip was incubated for 30 minutes, and then the microfluidic chip was washed with phosphate buffered saline containing Tween20; DAPI solution was then added into the microfluidic chip, and after incubation for 5 minutes, the chip was washed with phosphate buffered saline containing Tween20; and finally, fluorescence imaging was performed on the chip using an inverted fluorescence microscope, and the number of the tumor cells contained in the chip was counted. The statistical results are shown in FIG. 2.

From FIG. 2, the amount of the tumor cells in the healthy blood sample was 0 cells/ml, and the amount of the tumor cells detected by the microfluidic chip had a good linear relationship with the actual amount of the cells in the experimental group. The mean recovery rate for the tumor cells with the microfluidic chip was 93.4%, so the microfluidic chip was able to detect tumor cells in blood samples sensitively and accurately.

### Example 2

In the example of the present invention, a chimeric antigen receptor targeting EpCAM (referred to as EpCAM-CAR) was provided, and CAR-T cells (referred to as CAR-T) were constructed.

The molecular structure of EpCAM-CAR consists of a signal peptide, an antigen binding region, a hinge region, a transmembrane region and an intracellular costimulatory signal domain. The CAR plasmid composed of EpCAM-CAR and the backbone part of the third-generation lentiviral vector was constructed by OBiO Technology Corp., Ltd. using whole gene synthesis technology. See SEQ ID NO. 3 for the EpCAM-CAR sequence.

The Specific Packaging Method Was:

293T cells were inoculated in T75 culture flasks and cultured until the confluence was about 70%-80%, then the medium was replaced with equal volume of fresh medium to obtain the sample to be transfected. The number of the cells in the culture flask was controlled to 5×10⁶ cells/ml, the culture volume was 20 ml, and the medium used was DMEM medium containing 10% fetal bovine serum;

Tube A solution was prepared using 2 ml of Opti-MEM and 55 μl of Lipo3000; and Tube B solution was prepared using 2 ml of Opti-MEM, 46 μl of P3000, 18 μg of helper plasmid and 6 μg of EpCAM-CAR main plasmid;

The Tube A solution and the Tube B solution were mixed and incubated at room temperature for 15 minutes, and then the sample to be transfected was added thereinto and cultured for 48 hours;

after transfection for 48 hours, the supernatant was collected and centrifuged at 500 g for 10 minutes, the supernatant was filtered into a centrifuge tube and sealed, centrifuged at 10,000 g at 4° C. overnight to obtain a white virus precipitate; the white virus precipitate was extracted and dissolved with 200 μl of AIM-V medium, 2 ul of the solution was taken for measuring the titer according to the steps below, and the rest was stored at −80° C.

2 ul of resuspended virus supernatant was diluted by adding 198 ul of 1640 medium, and then 2 ul, 10 ul and 50 ul of the diluted virus solution were added to 3 wells in a 24-well plate at an amount of 2×10⁵ per well, respectively, and Polybrene helper virus was added at a final concentration of 5 ul/ml for infection for 48 hours to obtain Lentivirus. After virus infection, EpCAM-FITC labeled antigen was used to detect virus titer, and the titer was in a range of 2.5E+07-1.2E+08.

In the example of the present invention, CAR-T cells (referred to as CAR-T) were constructed by using the above lentivirus to infect human peripheral blood mononuclear cells (PBMC).

The Specific Construction Process is as Follows:

Human peripheral blood mononuclear cells (PBMC) were cultured in a medium consisting of AIM-V medium, 5% fetal bovine serum, 100 U/ml penicillin, 0.1 mg/ml streptomycin, and 300 IU/ml IL-2; T cells were activated by CD2/CD3/CD28 T cell activation and expansion kit from Miltenyi Company, that is, coated magnetic beads were mixed with the cells at a ratio of 1:2 so that the final cell density was 5×10⁶/ml/cm²; the mixture was then placed in an incubator at 37° C., 5% CO₂, for culture and stimulation for 48 hours; RetroNectin from Takara was diluted to 20 μg/ml to coat a non-tissue culture treated plate at 4 μg/cm², and then the plate was placed in a 4° C. refrigerator overnight. After T cell activation for 48 hours, the solution was centrifuged at 300 g for 5 minutes and the supernatant was removed, the T cells were resuspend in fresh medium, and transferred to the plate coated with RetroNectin from Takara, the above lentivirus was added and controlled at MOI=5, and the plate was placed in an incubator at 37° C., 5% CO₂, for culture; and also, T cells without lentivirus were prepared as a reference sample (referred to as unT); 24 hours after lentivirus was added, the solution was centrifuged at 300 g for 5 minutes, the supernatant was removed, and T cells were resuspended with medium to obtain CAR-T.

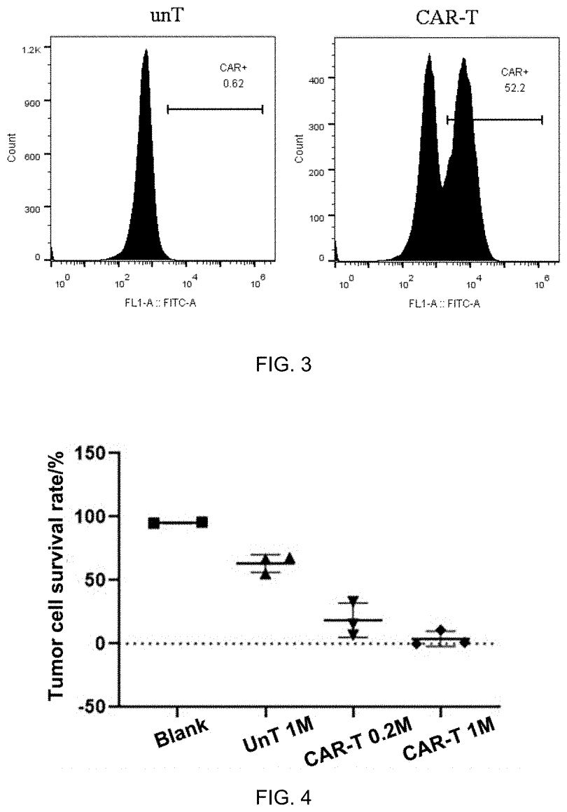

Further, 48 hours after lentivirus was added, a sample was taken and detected by flow cytometry for transduction rate, wherein EpCAM protein was used as the primary antibody, and anti-EpCAM-FITC from Biolegend Company was used as the secondary antibody, and a comparison graph of the flow cytometry analysis results of the reference sample and CAR-T as shown in FIG. 3 was obtained, which showed that CAR was successfully expressed on CAR-T.

### Example 3

This example proved that immune killer cells can eliminate CTCs in an in vitro simulation system, the example comprising:

a 24-well plate was divided for a healthy blood sample group, a blank control group (Blank group), a UnT group and a CAR-T group. The CAR-T group was divided into low-dose CAR-T group 1 and high-dose CAR-T group 2.

In the 24-well plate, 0.5 ml of whole blood was added in each of three wells 2-A, 2-B, and 2-C in the second column, four wells 3-A, 3-B, 3-C and 3-D in the third column and four wells 4-A, 4-B, 4-C and 4-D in the fourth column, and then 10 μl of human colorectal cancer cell suspension was added in each well and mixed uniformly to simulate the blood sample containing CTCs. The human colorectal cancer cell suspension was composed of HCT116 cells having a content of 1×10⁴ cells/ml and phosphate buffered saline. 10 microliters of phosphate buffered saline was added to well 2-D in the second column of the 24-well plate as a blank group, and the blood sample modeling of the in vitro CTC killing experiment was completed.

Then, effector cells were co-incubated with the blood samples in wells 2-A, 2-B, 2-C, 3-A, 3-B, 3-C, 3-D, 4-A, 4-B, 4-C, and 4-D, and the specific steps were:

Untransduced T cell (UnT) suspension was composed of UnT cells with a content of 1×10⁷ cells/ml and AIMV medium. The untransduced T cells were derived from human peripheral blood mononuclear cells (PBMC).

CAR-T cell suspension 1 and CAR-T cell suspension 2 consist of CAR-T cells and AIMV medium, wherein the content of CAR-T cells in CAR-T cell suspension 1 was 2×10⁵ cells/ml, the content of CAR-T cells in CAR-T cell suspension 2 was 1×10⁷ cells/ml. The CAR-T cells in CAR-T cell suspension 1 and CAR-T cell suspension 2 were obtained from Example 2.

0.1 ml of AIMV medium was added to each of wells 3-D and 4-D to form AIMV medium group, and 0.1 ml of UnT cell suspension was added to each of wells 2-C, 3-C and 4-C to form UnT group, in which the UnT concentration was 1M, 0.1 ml of CAR-T cell suspension 1 was added to each of wells 2-B, 3-B and 4-B to form low-dose CAR-T group 1, in which the CAR-T concentration was 0.02M, and 0.1 ml of CAR-T cell suspension 2 was added to each of wells 2-A, 3-A and 4-A to form high-dose CAR-T group 2, in which the CAR-T concentration was 1M.

The 24-well plate was placed in a cell incubator containing 5% CO₂ at 37° C., and cultured for 24 hours.

After the culture, the number of tumor cells contained in each group was counted using the chip detection steps provided in Example 1, and the statistical results of the experimental group data are shown in FIG. 4. The tumor cell content in the healthy blood sample was 0 cells/ml, and the mean tumor cell survival rates in the blood samples of the Blank group, UnT group, CAR-T group 1 and CAR-T group 2 were 95.4%, 63.2%, 18.7% and 4.0%, respectively. The survival rates of tumor cells in the blood samples of the CAR-T groups were significantly lower than those of the AIMV group and the UnT group, while the survival rate of tumor cells in the blood sample of the high-dose CAR-T group 2 was further decreased than that of the low-dose CAR-T group 1. The experiment results show that in this in vitro circulating tumor cell-killing model, CAR-T cells can significantly reduce the survival rate of tumor cells in the blood sample, and have a strong tumor killing ability, and with the concentration of CAR-T cells increased, the tumor-killing ability thereof in the blood sample was enhanced.

### Example 4

In this example, a first use of an animal model of tumor metastasis was provided, proving that CAR-T can inhibit tumor metastasis by killing CTCs in the blood sample.

Step S0 of the first use comprised: establishing a mouse model of human colorectal cancer hematogenous metastasis as an animal model of tumor metastasis by subcutaneous xenograft and tail vein injection. The specific steps were:

Cryopreserved human colorectal cancer cell suspension and several 6-week-old female M-NSG experimental mice weighted 18-22 g were provided; wherein, human colorectal cancer cell suspension 1 was composed of HCT116 cells with a content of 5×10⁷ cells/ml and phosphate buffered saline, and human colorectal cancer cell suspension 2 was composed of HCT116 cells with a content of 1×10⁷ cells/ml and phosphate buffered saline.

The human colorectal cancer cell suspension 1 was injected into each experimental mouse at a dose of 0.1 ml by subcutaneous injection on the right side, and the injection was completed within 3 seconds to ensure the injected dose and the activity of HCT116 cells.

After the tail vein injection was completed, the mice were raised under SPF condition, and the growth of the mice and the tumors were observed regularly. When the mean tumor volume was about 110 mm³, animals that were too large, too small or had irregular tumor shapes were eliminated.

The day on which the first administration was performed was recorded as Day 0. On Day 3, the human colorectal cancer cell suspension 2 was injected into each experimental mouse twice by tail vein injection, as a mouse model of human colorectal cancer blood metastasis. The injection dose of the human colorectal cancer cell suspension 2 was 0.1 ml each time, and the interval between each injection was 15 minutes. It should be noted that it was necessary to make sure that the needle tip was inside the tail vein, and the injection of human colorectal cancer cell suspension 2 was completed within 10 seconds to ensure the injected dose and the activity of HCT116-Luc cells.

On Day 5, in vivo fluorescence imaging was performed once on the mice, and the mean fluorescence value of the mouse tumor reached 5E5-5E6, completing the modeling of the mouse model of human colorectal cancer hematogenous metastasis.

Step 51 of the First Use Comprised:

Several mouse models of human colorectal cancer blood metastasis obtained after subcutaneous xenograft in step S0 of this example were randomly divided into a control group and an administration group; the administration group was divided into a UnT group and a CAR-T group, with 8 mice in each group, and totally 24 mice.

The administrations were performed on different groups on Day 0. Each experimental mouse in the control group was injected with 0.2 ml of normal saline through tail vein injection. Each experimental mouse in the UnT group was injected with 0.2 ml of UnT cell suspension at a dose of 3.57×10⁶ UnT cells per experimental mouse via tail vein injection. Each experimental mouse in the CAR-T group was injected with 0.2 ml of EpCAM-targeting CAR-T cell suspension at a dose of 3.57×10⁶ CAR-T cells per experimental mouse via tail vein injection. The CAR-T cells in the EpCAM-targeting CAR-T cell suspension were obtained from Example 2.

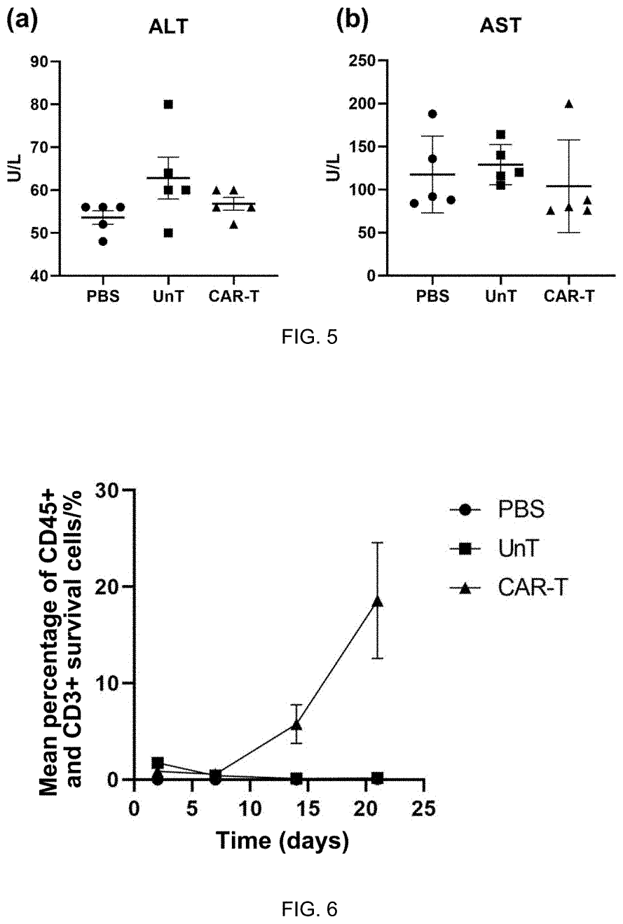

On Day 17, blood was collected from the intraocular canthus of the mice, and the alanine aminotransferase ALT (U/L) and aspartate aminotransferase AST (U/L) contents of the mice in each group were detected. As shown in FIG. 5, the mean alanine aminotransferase ALT (U/L) contents of the PBS group, the UnT group and the CAR-T group were 53.6±1.6, 62.8±4.9 and 56.8±1.5, respectively; and the aspartate aminotransferase AST (U/L) contents were 117.6±19.9, 129±10.4 and 104±24.1, respectively. There was no significant difference in ALT and AST among the groups, indicating that CAR-T showed no toxicity to liver at this dose.

On Day 21, blood was collected from the intraocular canthus of the mice, and the percentage of human CD45+ and human CD3+ T cells in the peripheral blood of the mice in each group was detected by flow cytometry (FACS). As shown in FIG. 6, the mean percentages of human CD45+ and human CD3+ survival in the peripheral blood of the mice in the PBS group and the UnT group were 0.06% and 0.16%, respectively, while the percentage of human CD45+ and human CD3+ survival in the peripheral blood of the mice in the CAR-T group was significantly higher, which was up to 18.56%. The mean percentages of CAR+ in the lymphocytes of the mice in the PBS group, the UnT group and the CAR-T group were 0.01%, 0.00% and 0.79%, respectively. The experiment results showed that compared with untransduced T cells, CAR-T had a longer survival time in peripheral blood.

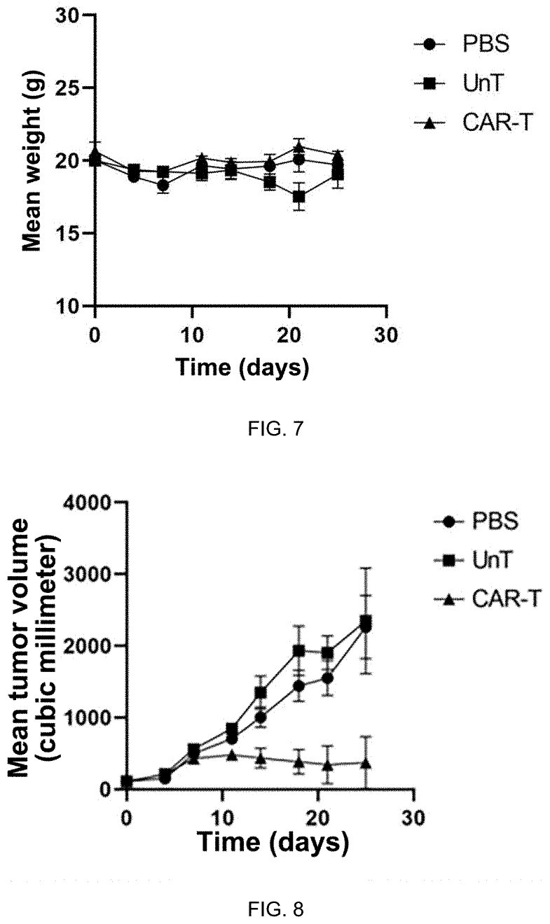

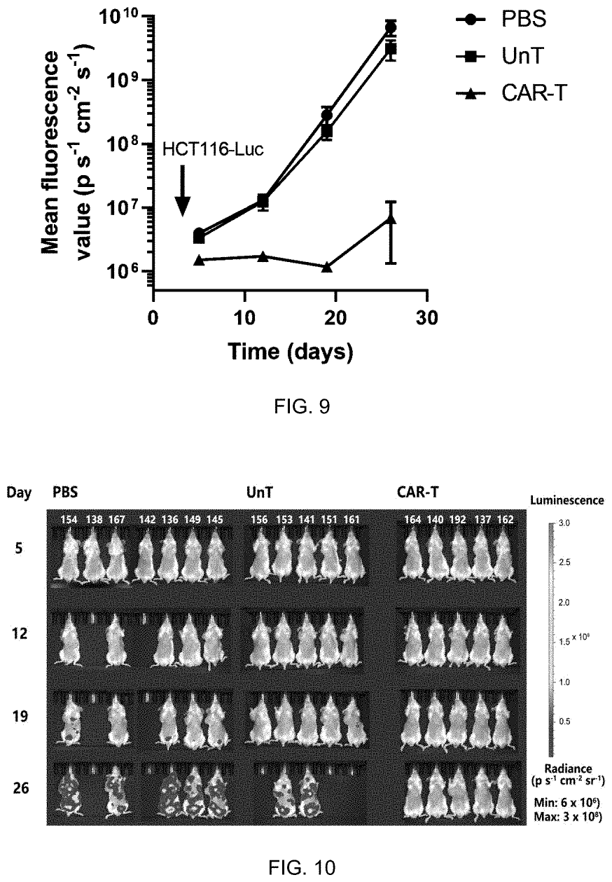

The mice were weighed twice a week, and the results are shown in FIG. 7. On Day 25, the weight growth rates of the mice in the control group, the UnT group and the CAR-T group were −1.62%, −4.75% and −1.15%, respectively. The mice in the CAR-T group could maintain their body weight well during the experiment, indicating that CAR-T did not show obvious toxicity at this dose.

The tumor diameters of the mice were measured twice a week, and the results are shown in FIG. 8. On Day 25, the mean tumor volume of the mice in the control group was 2260.46±441.97 mm³, and the mean tumor volume of the mice in the UnT group was 2347.27±737.55 mm³, and the tumor inhibition rate was −4.05%. The mean tumor volume of the mice in the CAR-T group was 373.12±360.01 mm³, and the tumor inhibition rate was 87.77%. The mean tumor volume in the CAR-T group was significantly lower than that in the control group, indicating that CAR-T had a significant tumor inhibitory effect at this dose.

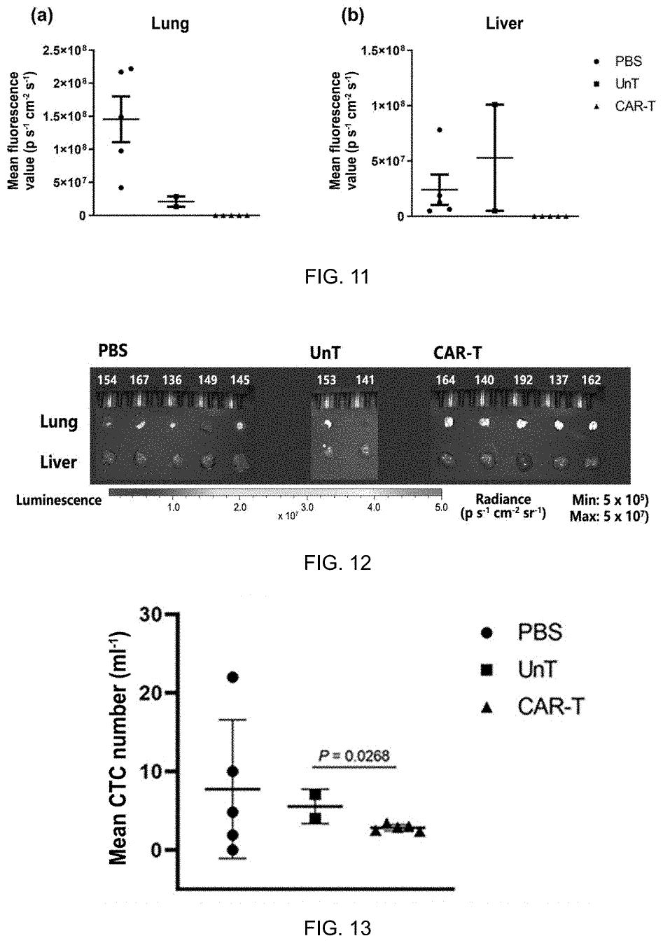

In vivo fluorescence imaging was performed once a week, and the results are shown in FIG. 10. As the feeding time prolonged, the fluorescence signals in the mice of the PBS group and the UnT group were significantly increased. There was no obvious change in the fluorescence signals of the mice in the CAR-T group. On Day 26, the mean tumor fluorescence value of the mice in the control group was 6.70×10⁹±1.79×10⁹ p/s, and the mean tumor fluorescence values of the mice in the UnT group and the CAR-T group were 3.10×10⁹±1.07×10⁹ p/s and 6.80×10⁸±5.47×10⁸ p/s, respectively. The mean tumor fluorescence value of the mice in the CAR-T group was significantly lower than those in the control group and the UnT group. At the end of the experiment, the mice were anticoagulated and euthanized according to experimental requirements. The mice were dissected and their lungs and livers were taken for ex vivo imaging. The results are shown in FIG. 11 and FIG. 12. The mean fluorescence value of the isolated livers of the mice in the control group was 2.41×10⁷±1.37×10⁷ p/s, while the mean fluorescence values of the isolated livers of the mice in the UnT and CAR-T groups were 5.28×10⁷±4.79×10⁷ p/s and 6.22×10⁴±3.21×10³ p/s, respectively. The mean fluorescence value of the isolated livers of the mice in the CAR-T group was significantly lower than those in the control group and the UnT group. The mean fluorescence value of the isolated lungs of the mice in the control group was 1.45×10⁸±3.46×10⁷ p/s, while the mean fluorescence values of the isolated lungs of the mice in the UnT group and the CAR-T group were 2.09×10⁷±7.72×10⁸ p/s and 7.59×10⁴±5.62×10³ p/s, respectively. The mean fluorescence value of the isolated lungs of the mice in the CAR-T group was significantly lower than those in the control group and the UnT group. The above experiments showed that CAR-T had a significant inhibitory effect on tumor metastasis at this dose.

Step S2 of the First Use Comprised:

On Day 26, blood was collected from the intraocular canthus of the mice, and the number of the tumor cells in the peripheral blood sample of each surviving experimental mouse in the control group and the experimental group was detected and counted by microfluidic chip technology.

The statistical results of the experiment are shown in FIG. 13. At the end of the experiment, the mean CTC content in the blood samples of the mice in the control group was 7.74/ml, while the mean CTC content in the blood samples of mice in the UnT group was 5.55/ml, and the mean CTC content in the blood samples of the mice in the CAR-T group was 2.84/ml. The mean number of the tumor cells in the peripheral blood samples of the mice in the CAR-T group was significantly lower than those in the control and UnT groups, which showed that CAR-T had a significant CTC killing effect in blood at this dose.

### Example 5

In this example, a second use of the animal model of tumor metastasis was provided, proving that CAR-T can inhibit tumor metastasis by killing CTCs in the blood sample.

Step S0 of the second use comprised: establishing a mouse model of human colorectal cancer hematogenous metastasis as an animal model of tumor metastasis via tail vein injection. The specific steps were:

Cryopreserved human colorectal cancer cell suspension and several 6-week-old female M-NSG experimental mice weighted 18-22 g, were provided; wherein, the human colorectal cancer cell suspension was composed of HCT116 cells with a content of 1×10⁷ cells/ml and phosphate buffered saline.

The human colorectal cancer cell suspension was injected into each experimental mouse twice via tail vein injection at a dose of 0.1 ml each time. The interval between each injection was 15 minutes. It should be noted that it was necessary to make sure that the needle tip was inside the tail vein, and the injection of human colorectal cancer cell suspension was completed within 10 seconds to ensure the injected dose and the activity of HCT116-Luc cells.

After the tail vein injection was completed, the mice were raised under SPF condition, and the mice were observed regularly by in vivo imaging of the mice and detecting the fluorescence value of tumor metastasis. When the mean fluorescence value of tumor metastasis reached 5E6-5E7 p/s, animals having too high or too low fluorescence values were eliminated, and the modeling of the mouse model of human colorectal cancer hematogenous metastasis was completed.

Step S1 of the Second Use Comprised:

Several mouse models of human colorectal cancer blood metastasis obtained through step S0 of the second use were randomly divided into a control group and an administration group; the administration group was divided into a UnT group and a CAR-T group, with 6 mice in each group, and totally 18 experimental mice.

The day on which the first administration was performed was recorded as Day 0. The administrations were performed on different groups on Day 0. Each experimental mouse in the control group was injected with 0.2 ml of normal saline through tail vein injection. Each experimental mouse in the UnT group was injected with 0.2 ml of untransduced T cell suspension at a dose of 8.93×10⁶ UnT cells per kilogram of experimental mouse via tail vein injection. Each experimental mouse in the CAR-T group was injected with 0.2 ml of EpCAM-targeting CAR-T cell suspension at a dose of 8.93×10⁶ CAR-T cells per experimental mouse via tail vein injection. The CAR-T cells in the EpCAM-targeting CAR-T cell suspension were obtained from Example 3.

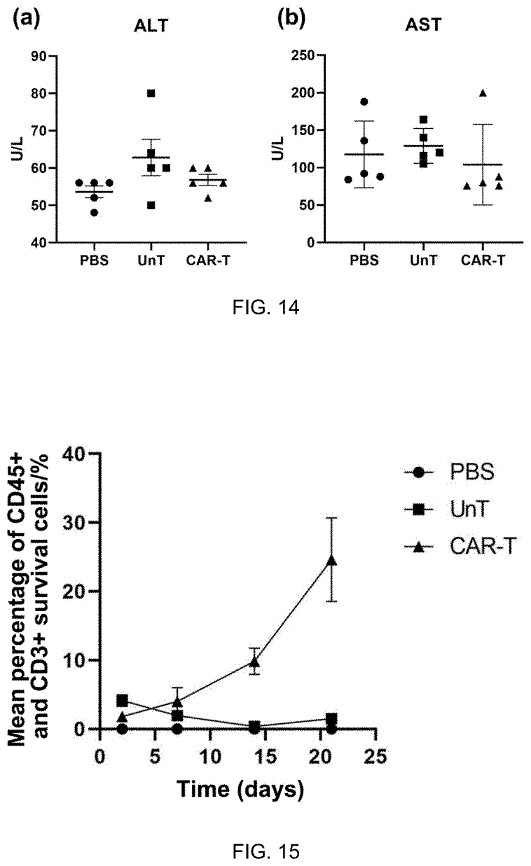

On Day 17, blood was collected from the intraocular canthus of the mice, and the alanine aminotransferase ALT (U/L) and aspartate aminotransferase AST (U/L) contents of the mice in each group were detected. As shown in FIG. 14, the mean ALT (U/L) contents of the PBS group, the UnT group and the CAR-T group were 52.3±1.7, 53.5±2.6 and 52.7±2.8, respectively; and the mean AST (U/L) contents were 95.8±7.0, 88.7±5.1 and 86.7±3.4, respectively. There was no significant difference in ALT and AST among the groups, indicating that CAR-T showed no toxicity to liver at this dose.

On Day 21, blood was collected from the intraocular canthus of the mice, and the percentages of human CD45+ and human CD3+ T cell survival in the peripheral blood and CAR+ in the lymphocytes of the mice in each group were detected by flow cytometry (FACS). The FACS detection results were shown in FIG. 15: the mean percentages of human CD45+ and human CD3+ survival in the peripheral blood of the mice in the PBS group, the UnT group and the CAR-T group were 0.00%, 1.52%, and 24.63%, respectively. On Day 21, the mean percentages of CAR+ in the lymphocytes of the mice in the PBS group, the UnT group and the CAR-T group were 0.00%, 0.00% and 2.79%, respectively. The experiment results showed that compared with UnT, CAR-T had a longer survival time in the body of mouse.

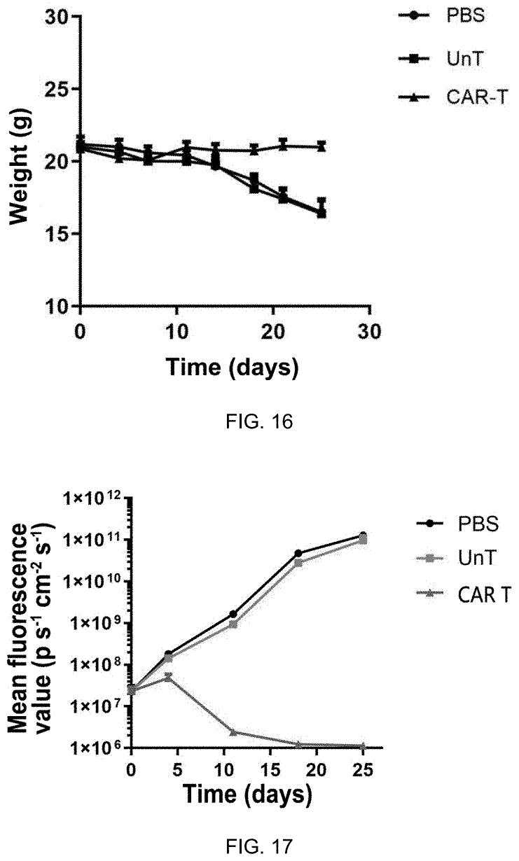

The mice were weighed twice a week, and the results are shown in FIG. 16. On Day 25, the weight growth rates of the mice in the PBS group, the UnT group and the CAR-T group were −22.03%, −21.90% and −0.56%, respectively. The mice in the CAR-T group could maintain their body weight well during the experiment, indicating that CAR-T did not show obvious toxicity at this dose.

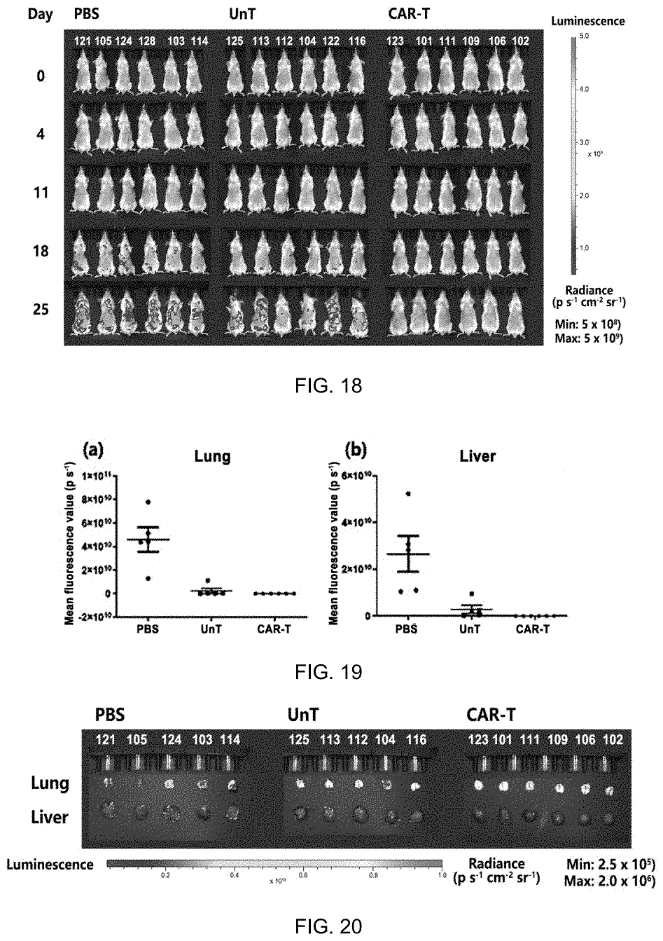

In vivo fluorescence imaging was performed once a week, and the results are shown in FIGS. 17 and 18. As the feeding time prolonged, the fluorescence signals in the mice of the PBS group and the UnT group were significantly increased, and there was no obvious change in the fluorescence signals of the mice in the CAR-T group. On Day 25, the mean tumor fluorescence value of the mice in the PBS group was 1.28E11±1.63E10 p/s, and the mean tumor fluorescence values of the mice in the UnT group and the CAR-T group were 9.73E10±2.66E10 p/s and 1.14E6±5.30E4 p/s, respectively. The mean tumor fluorescence value of the mice in the CAR-T group was significantly lower than that in the PBS group, which showed that CAR-T had a significant inhibitory effect on tumor metastasis at this dose.

At the end of the experimental plan, on Day 27, the mice were euthanized according to experimental requirements. The mice were dissected and their lungs and livers were taken for ex vivo imaging. The results are shown in FIG. 19 and FIG. 20. The mean fluorescence values of the isolated lungs of the mice in the PBS group, the UnT group and the CAR-T group were 4.66E10±1.34E10, 2.32E9±2.18E9, and 8.41E4±3.76E3, respectively; and the mean fluorescence values of the isolated livers of the mice were 2.65E10±7.71E9, 2.84E9±1.73E9 and 1.74E5±2.92E4, respectively. The mean fluorescence values of the isolated lungs and livers of the mice in the CAR-T group were significantly lower than those in the PBS group, which showed that CAR-T had a significant inhibitory effect on tumor metastasis at this dose.

Step S2 of the Second Use Comprised:

On Day 27, blood was collected from the intraocular canthus of the mice, and the number of the tumor cells in the peripheral blood sample of each surviving experimental mouse in the control group and the experimental group was detected and counted by microfluidic chip technology.

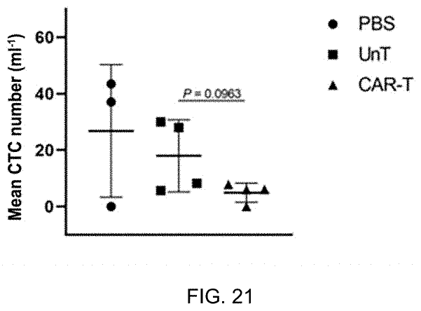

The statistical results of the experiment are shown in FIG. 21. At the end of the experiment, the mean CTC content in the blood samples of the mice in the control group was 26.87/ml, while the mean CTC content in the blood samples of mice in the UnT group was 18/ml, and the mean CTC content in the blood samples of the mice in the CAR-T group was 4.98/ml. The mean number of the tumor cells in the peripheral blood samples of the mice in the CAR-T group was significantly lower than those in the control and UnT groups, which showed that CAR-T had a significant CTC killing effect in blood at this dose.

Although the examples of the present invention have been described in detail above, it will be apparent to those skilled in the art that various modifications and changes can be made to these examples. However, it should be understood that such modifications and changes are within the scope and spirit of the invention as set forth in the appended claims. Furthermore, the invention described herein may have other embodiments and may be implemented or realized in various ways.

What is claimed:

1. Use of immune killer cells targeting circulating tumor cells in the treatment of solid tumors, comprising:
S0: providing a subject and the immune killer cells, the subject's peripheral blood comprising the circulating tumor cells; and
S1: injecting an effective dose of the immune killer cells into the peripheral blood of the subject, and slowing down or blocking metastasis of the solid tumors through specifically recognizing the circulating tumor cells and killing or eliminating the circulating tumor cells by the immune killer cells.

2. The use of the immune killer cells targeting circulating tumor cells in the treatment of solid tumors according to claim 1, wherein the immune killer cells are genetically modified immune cells.

3. The use of the immune killer cells targeting circulating tumor cells in the treatment of solid tumors according to claim 2, wherein the immune cells are at least one of T cells, NK cells, NKT cells, dendritic cells, macrophages and B cells.

4. The use of the immune killer cells targeting circulating tumor cells in the treatment of solid tumors according to claim 2, wherein the genetically modified immune cells are obtained by genetically modifying the immune cells with any one of chimeric antigen receptors and T cell receptors.

5. The use of immune killer cells targeting circulating tumor cells in the treatment of solid tumors according to claim 4, wherein the chimeric antigen receptor comprises an extracellular recognition region, a hinge region, a transmembrane region and an intracellular signal region, and the extracellular recognition region specifically recognizes any one of EpCAM, c-MET, CD47, Vimentin, E-cadherin, Cytokeratins, Zonula occludens, ESPR1, N-cadherin, Twist1, ZEB1, FGFR2IIIc, PLS3, ALDH1, CD44, GD2, GD3, Claudin18.2, Claudin6, and GD1a.

6. The use of the immune killer cells targeting circulating tumor cells in the treatment of solid tumors according to claim 5, wherein a sequence of the hinge region is derived from at least one of CD8α, CD28, 4-1BB, ICOS, OX40, CD40, CD80 and IgG; a sequence of the transmembrane region is derived from at least one of CD8α, CD28, 4-1BB, ICOS, OX40, CD40 and CD80; and a sequence of the intracellular signal region is derived from at least one of CD8α, CD28, 4-1BB, ICOS, OX40, CD40, CD80, DAP10, DAP12, CD3 and CD3e.

7. The use of the immune killer cells targeting circulating tumor cells in the treatment of solid tumors according to claim 4, wherein the genetically modified immune cells are obtained by genetically modifying T cells with chimeric antigen receptors.

8. The use of the immune killer cells targeting circulating tumor cells in the treatment of solid tumors according to claim 1, wherein the subject is an animal model of solid tumor metastasis or a patient with metastases, and the tumor metastasis patterns of both the animal model of solid tumor metastasis and the patient with metastases are hematogenous metastases.

9. The use of the immune killer cells targeting circulating tumor cells in the treatment of solid tumors according to claim 8, wherein the step S0 further comprises introducing exogenous circulating tumor cells into a body of an experimental animal via any one of orthotopic transplantation, intravenous injection and subcutaneous transplantation, so as to establish the animal model of solid tumor metastasis.

10. The use of the immune killer cells targeting circulating tumor cells in the treatment of solid tumors according to claim 9, wherein the step S1 is performed after the exogenous circulating tumor cells are introduced into the body of the experimental animal, and after obtaining a peripheral blood sample of the experimental animal and detecting that it contains the circulating tumor cells.

11. The use of the immune killer cells targeting circulating tumor cells in the treatment of solid tumors according to claim 9, wherein the step S1 is performed after the exogenous circulating tumor cells are introduced into the body of the experimental animal to form tumor tissue.

12. The use of the immune killer cells targeting circulating tumor cells in the treatment of solid tumors according to claim 9, wherein in the step S1, a cell suspension containing the immune killer cells is injected into the body of the experimental animal via intravenous injection, and the amount of the immune killer cells in the cell suspension injected each time is 1×10⁴/Kg−1×10⁸/Kg.

13. The use of the immune killer cells targeting circulating tumor cells in the treatment of solid tumors according to claim 1, wherein the step S1 further comprises applying auxiliary medical means to the subject to effectively eliminate the solid tumor.
